(19) United States
(12) Patent Application Publication
(10) Pub. No.: US 2011/0313214 A1
(43) Pub. Date: Dec. 22, 2011

(54) Hydro(chloro)fluoroolefins and Method for Preparation Thereof

(75) Inventor: Jean-Luc Dubois, Millery (FR)

(73) Assignee: Arkema France, Colombes (FR)

(21) Appl. No.: 13/003,426

(22) PCT Filed: Jul. 9, 2009
(86) PCT No.: PCT/IB09/06199

§ 371 Date: Apr. 1, 2011

(30) Foreign Application Priority Data
Jul. 10, 2008 (FR) 0803941

(51) Int. Cl. C07C 21/18; C12P 1/00

(52) U.S. Cl. 570/136; 570/135; 570/137; 435/41

(57) ABSTRACT

A subject of the invention is a compound of formula (I) XCF_{z}R_{3-z} in which: in which X represents a branched or linear unsaturated hydrocarbon radical having up to 5 carbon atoms, unsubstituted or substituted, R represents Cl, F, Br, I or H, Z is equal to 1, 2 or 3, and the bio-carbon content of which is at least 1%.

A subject of the invention is also processes for the preparation of this compound.

### FIELD OF THE INVENTION

A subject of the invention is hydro(chloro)fluoroolefins and their process of preparation from renewable raw materials.

### TECHNOLOGICAL BACKGROUND

Hydro(chloro)fluoroolefins or H(C)FOs are known in particular for their properties as refrigerants and heat-transfer fluids, extinguishers, propellants, foaming agents, swelling agents, gaseous dielectrics, a polymerization or monomer medium, support fluids, abrasive agents, drying agents and fluids for an energy production unit. Unlike CFCs and HCFCs, which are potentially dangerous for the ozone layer, HFOs do not contain chlorine and therefore do not pose a problem for the ozone layer.

These compounds are synthesized from hydrocarbon compounds, i.e. originating from the oil industry. Whilst HFOs are not dangerous to the ozone layer, the eco-balance of their production is not perfect, in particular the CO₂ balance. These compounds have replaced HFCs due to a lower GWP (Global Warming Potential). However, current production processes for H(C)FOs still contribute to global warming.

A subject of the invention is therefore reducing global warming during production of the H(C)FOs, by reducing the greenhouse gas emissions linked with their production.

A subject of the invention is therefore improving the carbon footprint (cumulative greenhouse gas emissions linked with the production of raw materials and with the production process) of H(C)FOs.

### SUMMARY OF THE INVENTION

A subject of the invention is a compound of formula (I)

XCF_{z}R_{3-z}

in which:
X represents a unsubstituted or substituted, branched or linear unsaturated hydrocarbon radical having up to 5 carbon atoms,
R represents Cl, F, Br, I or H,
Z is equal to 1, 2 or 3,
   and the bio-carbon content of which is at least 1%.

According to an embodiment of the invention, the bio-carbon content is greater than 5%, preferably greater than 10%, preferably greater than 25%, preferably greater than or equal to 33%, preferably greater than 50%, preferably greater than or equal to 66%, preferably greater than 75%, preferably greater than 90%, preferably greater than 95%, preferably greater than 98%, preferably greater than 99%, advantageously substantially equal to 100%.

According to an other embodiment of the invention, X is a radical which is unsubstituted or substituted by at least one atom, preferably 1 to 9 atoms, advantageously 1 to 6 atoms, chosen from Cl, F, Br and I.

According to a further embodiment of the invention, the compound according to the invention corresponds to formula (II):

R₂C═CRR′

in which
R′ represents (CR₂)ₙY
Y represents CRF₂
each R independently has the meaning given in claims 1 to 3
n is equal to 0, 1, 2 or 3.

According to an embodiment, n is equal to 0 or 1.

According to an embodiment, Y represents CF₃.

According to an embodiment, R represents Cl, F or H.

According to an embodiment, the compound is chosen from the group consisting of tetrafluoropropenes (1234 series), pentafluoropropenes (1225 series) and chlorotrifluoropropenes (1233 series).

According to an embodiment, the compound is chosen from the group consisting of

2,3,3,3-tetrafluoro-1-propene (1234yf),

1,3,3,3-tetrafluoro-1-propene (1234ze, cis and trans),

1,2,3,3,3-pentafluoropropene (1225ye, cis and trans),

1,1,3,3,3-pentafluoropropene (1225zc),

3,3,3-trifluoro-2-chloro-1-propene (1233xf), and

3,3,3-trifluoro-1-chloro-1-propene (1233zd).

A further subject of the invention is a process for the preparation of the compound according to the invention, comprising the stage of providing one or more carbon-containing chains comprising one or more carbon atoms having a biocarbon content of at least 1%, and the conversion by synthesis to the sought halogenated compound.

According to an embodiment, the said stage of providing comprises a stage of production of alcohol, in particular of methanol, ethanol, n-propanol and n-butanol.

According to an embodiment, the stage of production of alcohol is carried out by fermentation of the biomass.

According to an embodiment of the invention, the stage of production of alcohol comprises the following sub-stages:
(i) production of methane from biomass,
(ii) steam reforming of the latter to a synthesis gas or production of synthesis gas by direct gasification of biomass and
(iii) production of alcohol from this synthesis gas.

According to an embodiment, the stage of production of alcohol comprises the following sub-stages:
(i) production of methane from biomass,
ii) direct oxidation to methanol.

According to an other embodiment, the alcohol is converted to olefin.

According to a further embodiment, the methanol is converted to DME which is then dehydrated to olefin.

According to an embodiment, the alcohol is converted to a saturated halogenated compound.

According to an embodiment, the methanol is converted to the compound CHₘClₙF₄₋ₘ₋ₙ with each m being able to represent an integer from 0 to 3 and each n being able to represent an integer from 0 to 4 and m+n being at most equal to 4.

According to an embodiment, the compound CHₘClₙF₄₋ₘ₋ₙ reacts with at least one olefin.

According to an embodiment, the synthesis stage comprises at least one chlorination stage followed by at least one partial or total fluorination stage.

According to an embodiment, the synthesis stage comprises at least one dehydrohalogenation stage.

### DETAILED DESCRIPTION OF EMBODIMENTS

The invention uses products of natural origin as starting products. The carbon in a biomaterial originates from the photosynthesis of plants and therefore from atmospheric CO₂. The degradation (by degradation is also meant end-of-life combustion/incineration) of these materials to CO₂ does not therefore contribute to warming since there is no increase in the carbon emitted into the atmosphere. The CO₂ balance of biomaterials is therefore significantly better and contributes to a reduction in the carbon footprint of the products obtained (only the energy used for production is to be taken into account). By contrast, a material of fossil origin also degrading to CO₂ will contribute to an increase in the level of CO₂ and therefore to global warming.

The compounds according to the invention will therefore have a carbon footprint which will be better than that of compounds obtained from a fossil source.

The invention therefore also improves the eco-balance during the production of H(C)FOs.

The term “bio-carbon” indicates that the carbon is of natural origin and comes from a biomaterial, as indicated below. The biocarbon content and biomaterial content are expressions denoting the same value.

A material of renewable origin or biomaterial is an organic material in which the carbon originates from CO₂ fixed recently (on a human scale) by photosynthesis from the atmosphere. On land, this CO₂ is captured or fixed by plants. In the sea, the CO₂ is captured or fixed by bacteria or vegetation or microscopic algae carrying out photosynthesis. A biomaterial (of 100% natural carbon origin) has an isotopic ¹⁴C:¹²C ratio greater than 10⁻¹², typically of the order of 1.2×10⁻¹², whereas a fossil material has a ratio of zero. In fact, the ¹⁴C isotope is formed in the atmosphere and is then integrated by photosynthesis, over a time scale of a few tens of years at most. The half-life of ¹⁴C is 5730 years. Therefore the materials originating from photosynthesis, namely plants in general, necessarily have a maximum ¹⁴C isotope content.

Determination of the biomaterial content or biocarbon content is carried out by applying the standards ASTM D 6866 (ASTM D 6866-06) and ASTM D 7026 (ASTM D 7026-04). The standard ASTM D 6866 has the subject “Determining the Biobased Content of Natural Range Materials Using Radiocarbon and Isotope Ratio Mass Spectrometry Analysis”, whereas the standard ASTM D 7026 has the subject “Sampling and Reporting of Results for Determination of Biobased Content of Materials via Carbon Isotope Analysis”. The second standard refers to the first in its first paragraph.

The first standard describes a test for measuring the ratio of a sample and compares it with the ¹⁴C:¹²C ratio of a reference sample of 100% renewable origin, in order to produce a relative percentage of C of renewable origin in the sample. The standard is based on the same concepts as dating with the ¹⁴C, but without applying the dating equations.

The ratio thus calculated is denoted as the “pMC” (percent Modern Carbon). If the material to be analyzed is a mixture of biomaterial and fossil material (with no radioactive isotope), then the pMC value obtained is directly correlated to the quantity of biomaterial present in the sample. The reference value used for the ¹⁴C dating is a value dating from the 1950s. This year was chosen due to the existence of nuclear tests in the atmosphere which introduced large quantities of isotopes into the atmosphere after this date. The 1950 reference corresponds to a pMC value of 100. Taking the thermonuclear tests into account, the current value to be adopted is approximately 107.5 (which corresponds to a correction factor of 0.93). The radioactive carbon signature of a plant growing now is therefore 107.5. Signatures of 54 pMC and 99 pMC therefore correspond to a quantity of biomaterial in the sample of 50% and 93%, respectively.

The standard ASTM D 6866 proposes three techniques for 7 measuring the ¹⁴C isotope content:
LSC (Liquid Scintillation Counting) spectrometry. This technique consists of counting “beta” particles originating from the disintegration of the ¹⁴C. The beta radiation originating from a sample of known mass (known number of C atoms) is measured over a certain period of time. This “radioactivity” is proportional to the number of atoms of ¹⁴C, which can thus be determined. The ¹⁴C present in the sample emits β rays, which on contact with the scintillating liquid (scintillator) produce photons. These photons have different energies (comprised between 0 and 156 keV) and form what is called a ¹⁴C spectrum. According to two variants of this method, the analysis relates either to the CO₂ previously produced by the carbon-containing sample in a suitable absorbing solution, or to benzene after prior conversion of the carbon-containing sample to benzene. The standard ASTM D 6866 therefore gives two methods, A and C, based on this LSC method.
AMS/IRMS (Accelerated Mass Spectrometry coupled with Isotope Radio Mass Spectrometry). This technique is based on mass spectrometry. The sample is reduced to graphite or gaseous CO₂, and analyzed in a mass spectrometer. This technique uses an accelerator and a mass spectrometer to separate the ¹⁴C ions from the ¹²C ions and thus determine the ratio of the two isotopes.

The compounds according to the invention originate at least in part from biomaterial and therefore have a biomaterial content of at least 1%. This content is advantageously higher, in particular up to 100%. The compounds according to the invention can therefore comprise 100% bio-carbon or by contrast result from a mixture with material of fossil origin.

According to an embodiment, ¹⁴C/¹²C isotope ratio is comprised between 0.2×10⁻¹² and 1.2×10⁻¹².

The compounds according to the invention are, as indicated above, hydro(chloro)-fluoroolefins of formula I (XCF_{z}R_{3-z}), preferably of formula II (R₂C═CRR′).

In order to produce biocarbon-based H(C)FOs, in a first phase a non-halogenated carbon compound is produced. In a second phase, this non-halogenated compound is subjected to chlorination reactions, then fluorination or direct fluorination (and optionally chlorination) reactions. Coupling, pyrolysis, elimination and addition reactions are also possible. The H(C)FOs according to the invention are obtained in this way. The following reactions can be mentioned as examples of reactions capable of producing the biocarbon based non-halogenated compounds.

The production of methane from biomass is known. The biogas methane results from the methanization or anaerobic digestion of fermentable waste. Common sources are discharges, the selective collection of putrescible waste (optionally by the use of digesters), treatment plant sludge, livestock effluents, food industry effluents or a lake (for example Lake Kivu), etc. The biogas contains a majority proportion of methane.

This methane then undergoes an SMR (Steam Methane Reforming) reaction. On completion of this reaction a mixture of CO and hydrogen is obtained in a variable ratio (typically approximately 2 to 3), also called synthesis gas or syngas. This syngas is used for example for the production of hydrocarbides by the Fischer-Tropsch reaction, hydrocarbides which can then be converted to various products, in particular olefins, by standard upgrading reactions. This syngas can also, depending on the H₂:CO ratio, and/or depending on the catalysts used, be converted to methanol or higher alcohols.

It is also possible to provide the direct gasification of biomass to synthesis gas or syngas, by reaction of the biomass carbon in the presence for example of oxygen (pure or atmospheric) or of water, at a more or less high temperature (for example from 800 to 1000° C.) at a pressure for example close to atmospheric pressure.

Regarding the conversion of the syngas to methanol or to higher alcohol, reference can be made to “Procédés de pétrochimie, IFP, ENSPM”, 1985, 2^{nd} edition, pp 90-104 and to “Fundamentals of Industrial Catalytic Processes”, Wiley, 2^{nd} edition, 6.4.8.

It is also possible to carry out direct (controlled) oxidation of methane directly to methanol.

The ethanol or a higher alcohol such as propanol or butanol can also be produced directly from biomass. A biomass is fermented using a yeast (for example Saccharomyces Cerevisiae) or a bacterium (for example Zymomonas or Clostrodium). Such processes are known to a person skilled in the art. The starting biomass is variable, in particular depending on the microorganism used, and it can be optionally hydrolyzed to elementary monosaccharides.

The methanol obtained from the biomass can then be converted to dimethylether (DME). The DME is then dehydrated in order to produce lower olefins, typically ethylene and propylene. This reaction is standard and well known to a person skilled in the art. Reference can be made to the following documents: “Heterogeneous catalysts in Industrial Practice”, Charles N. Satterfield, Second Edition, McGraw-Hill Inc, 7.7.9 Methanol to gasoline, p. 255; UOP “UOP/HYDRO MTO Process Methanol to Olefins Conversion”, 2007, UOP 4522-3 UOP-PTE 0708-002; Lurgi, “Methanol-To-Propylene-MTP®”, commercial brochure, pp 1-4.

The higher alcohols such as ethanol, propanol and butanol can then be dehydrated in a standard manner in order to obtain olefins by elimination of water.

The starting biomass can be a lignocellulosic biomass (wood, sugar cane, straw, etc.) or a glucide biomass (cereals, beet, etc.) which can be easily hydrolyzed.

The olefins thus obtained can then be converted by fluorination and/or chlorination reactions known to a person skilled in the art.

For example, starting from ethylene, it is possible to obtain vinyl chloride and dichloroethane by chlorination (see J. C LANET's Procédé CVM Chlorure de Vinyle Monomère). Starting from ethylene, the process comprising the reaction of tetrachloromethane with ethylene in order to produce 1,1,1,3-tetrachloropropane may also be mentioned. This derivative can then be converted to 3,3,3-trifluoro-1-propene by fluorination (optionally in the presence of a catalyst) and dehydrohalogenation reactions. The 3,3,3-trifluoro-1-propene can also be converted by chlorination with Cl₂ to 1,1,1-trifluoro-2,3-dichloro-propane. The latter compound can also undergo a dehydrochlorination reaction to 3,3,3-trifluoro-2-chloro-1-propene, which can then be reacted with HF in order to produce 3,3,3,2-tetrafluoro-2-chloro-propane. This compound can then be subjected to a dehydrochlorination reaction in order to produce 2,3,3,3-tetrafluoro-1-propene. 3,3,3-trifluoro-2-chloro-1-propene can also be obtained from the chlorine derivative 1,1,2,3-tetrachloro-1-propene (1230xa).

The ethylene can also undergo a fluorination reaction in order to produce the monofluoroethylene or difluoroethylene derivative or also other fluorinated derivatives.

Monofluoroethylene can react with CF₃Cl in order to produce the compound of formula CF₃CH₂CHFCl, which is then dehydrohalogenated (e.g. on potash) to produce 1,3,3,3-tetrafluoro-1-propene.

Difluoroethylene can react with a compound CHFCl₂, in order to produce a compound of formula CHClFCH₂CClF₂, which can then be exposed to conditions of dehydrohalogenation and/or fluorination in order to produce compound 1,3,3,3-tetrafluoro-1-propene.

Starting from propylene, there may be mentioned the reaction comprising the reaction with chlorine in order to produce the derivative CCL₃CHCLCH₂CL (catalyst Au/TiO2), which is fluorinated to produce CF₃CHCLCH₂F, which is then dehydrochlorinated in order to produce the compound 1,3,3,3-tetrafluoro-1-propene.

1,3,3,3-tetrafluoro-1-propene and 2,3,3,3-tetrafluoro-1-propene can be prepared by dehydrofluorination from 1,1,1,2,3-pentafluoropropane or by dehydrochlorination from 1,1,1,3-tetrafluoro-2-chloro-propane.

2,3,3,3-tetrafluoro-1-propene can also be prepared by dehydrofluorination starting from 1,1,1,2,2-pentafluoropropane (the latter compound can be obtained from the corresponding pentachlorinated compound via trichloroacetone or by successive stages of chlorination and fluorination via the intermediate 2,2-difluoropropane.

The preparation of pentafluoro-propene by dehydro-halogenation of hexafluoropropane may also be mentioned.

The hexafluoropropane and/or hexafluoropropene synthesis routes are standard and known to a person skilled in the art (in particular starting from difluoromonochloromethane).

The reactions referred to in the document Knunyants et al., (Journal of the USSR Academy of Sciences, Chemistry Department, “Reactions of fluoro-olefins”, report 13, “Catalytic hydrogenation of perfluoro-olefins”, 1960) may also be mentioned.

In a general manner, it is possible to carry out fluorination and/or chlorination of olefins, optionally producing a saturated compound which can undergo dehydrohalogenation reactions in order to produce the sought olefin.

It is possible to prepare chlorinated compounds from methanol, by reaction of HCl with methanol, which produces monochloromethane and water. The monochloromethane can then in turn be converted to tetrachloromethane by the action of Cl₂. Tetrachloromethane is an intermediate known in the chemistry relating to halogenated olefins. The chlorinated derivatives of methane can be fluorinated using hydrofluoric acid, in order to produce more or less fluorinated derivatives of methane (also containing or not containing chlorine atoms). These chlorinated and/or fluorinated methane compounds can be subjected for example to coupling or addition reactions.

1-22. (canceled)

23. A compound of formula (I):
XCF_{z}R_{3-z}
wherein:
X represents a substituted or unsubstituted, linear or branched, unsaturated hydrocarbon radical having up to 5 carbon atoms,
R represents Cl, F, Br, I or H,
z is equal to 1, 2, or 3, and
said compound comprises a bio-carbon content of at least 1%.

24. The compound of claim 23, wherein the bio-carbon content is greater than 5%.

25. The compound of claim 24, wherein the bio-carbon content is greater than 10%.

26. The compound of claim 25, wherein the bio-carbon content is greater than 50%.

27. The compound of claim 26, wherein the bio-carbon content is greater than 75%.

28. The compound of claim 27, wherein the bio-carbon content is greater than 90%.

29. The compound of claim 28, wherein the bio-carbon content is greater than 98%.

30. The compound of claim 29, wherein the bio-carbon content is substantially equal to 100%.

31. The compound of claim 23, wherein X is substituted by at least one halogen atom comprising Cl, F, Br, I, or a combination thereof.

32. The compound of claim 31, wherein X is substituted by 2 to 9 of said halogen atoms.

33. The compound of claim 32, wherein X is substituted by 2 to 6 of said halogen atoms.

34. The compound of claim 23, wherein said compound comprises a compound of formula (II):
R₂C═CRR′
wherein:
R′ represents (CR₂)ₙY
Y represents CRF₂
each R independently comprises Cl, F, Br, I or H, and
n is equal to 0, 1, 2 or 3.

35. The compound of claim 34, wherein n is equal to 0 or 1.

36. The compound of claim 34, wherein Y represents CF₃.

37. The compound of claim 23, wherein R represents Cl, F, or H.

38. The compound of claim 23, wherein said compound comprises a tetrafluoropropene, a pentafluoro-propene, or a chlorotrifluoropropene.

39. The compound of claim 23, wherein said compound comprises:
2,3,3,3-tetrafluoro-1-propene (1234yf),
1,3,3,3-tetrafluoro-1-propene (1234ze, cis and trans),
1,2,3,3,3-pentafluoropropene (1225ye, cis and trans),
1,1,3,3,3-pentafluoropropene (1225zc),
3,3,3-trifluoro-2-chloro-1-propene (1233xf), or
3,3,3-trifluoro-1-chloro-1-propene (1233zd).

40. The compound of claim 23, wherein said compound has a ¹⁴C/¹²C isotope ratio ranging from 0.2×10⁻¹² to 1.2×10⁻¹².

41. A process for preparation of a compound of formula (I):
XCF₂R_{3-z}
wherein:
X represents a substituted or unsubstituted, linear or branched, unsaturated hydrocarbon radical having up to 5 carbon atoms,
R represents Cl, F, Br, I or H,
z is equal to 1, 2, or 3, and
said compound comprises a bio-carbon content of at least 1%;
said process comprising the steps of:
providing one or more chains comprising one or more carbon atoms, wherein said chains have a biocarbon content of at least 1%, and
converting said chains into said compound by chemical synthesis.

42. The process of claim 41, wherein said providing step comprises producing an alcohol.

43. The process of claim 42, wherein said alcohol comprises methanol, ethanol, n-propanol, n-butanol, or a combination thereof.

44. The process of claim 42, wherein said alcohol is produced by fermentation of a biomass.

45. The process of claim 42, wherein producing the alcohol comprises the steps of:
i) producing methane from a biomass,
ii) steam reforming said methane to a synthesis gas, or producing the synthesis gas by direct gasification of the biomass, and
iii) producing the alcohol from the synthesis gas.

46. The process of claim 42, wherein producing the alcohol comprises the steps of:
i) producing methane from a biomass, and
ii) oxidizing said methane to methanol.

47. The process of claim 42, wherein said providing step further comprises converting the alcohol to an olefin.

48. The process of claim 47, wherein the step of converting the alcohol to olefin comprises converting methanol to DMF and dehydrating the DMF to said olefin.

49. The process of claim 42, further comprising converting the alcohol to a saturated halogenated compound.

50. The process of claim 49, wherein the saturated halogenated compound comprises a compound of formula:
CHₘClₙF₄₋ₘ₋ₙ
wherein:
m is 0, 1, 2, or 3,
n is 0, 1, 2, 3, or 4, and
m+n is at most equal to 4.

51. The process of claim 50, further comprising reacting the saturated halogenated compound with at least one olefin.

52. The process of claim 41, wherein converting said chains into said compound by chemical synthesis comprises performing at least one chlorination step followed by at least one partial or total fluorination step.

53. The process of claim 41, wherein converting said chains into said compound by chemical synthesis comprises at least one dehydrohalogenation step.
